(19) United States
(12) Patent Application Publication
(10) Pub. No.: US 2024/0001015 A1
(43) Pub. Date: Jan. 4, 2024

(54) DIALYSATE FILTER

(71) Applicant: W. L. Gore & Associates, Inc., Newark, DE (US)

(72) Inventors: James R. Hanrahan, Newark, DE (US); Kevin J. Mabe, Elkton, MD (US); Shailesh P. Vidhate, Middletown, DE (US)

(21) Appl. No.: 18/265,013

(22) PCT Filed: Dec. 1, 2021
(86) PCT No.: PCT/US2021/061379

§ 371 Date: Jun. 2, 2023

Related U.S. Application Data
(60) Provisional application No. 63/120,316, filed on Dec. 2, 2020.

(51) Int. Cl. A61M 1/16; A61M 1/36; B01D 39/16

(52) U.S. Cl. CPC A61M 1/1696; A61M 1/3687; A61M 1/3679; B01D 39/16; A61M 2209/088; B01D 2239/0407; B01D 2239/065

(57) ABSTRACT

Various aspects of the present disclosure are directed toward apparatuses, systems, and methods that include a filter for removal of waste products from a dialysate. The filter may include multiple layers of one or more filter materials configured to allow dialysate to flow through the multiple layers.

### CROSS-REFERENCE TO RELATED APPLICATION

This application is a national phase application of PCT Application No. PCT/US2021/061379, internationally filed on Dec. 1, 2021, which claims the benefit of Provisional Application No. 63/120,316, filed Dec. 2, 2020, both of which are incorporated herein by reference in their entireties for all purposes.

### FIELD

The present disclosure relates generally to apparatuses, systems, and methods directed toward filtering and removing of waste. More specifically, the disclosure relates to apparatuses, systems, and methods directed toward a filter for use in dialysis waste streams.

### BACKGROUND

Dialysis such as hemodialysis and peritoneal dialysis are commonly to treat loss of kidney function. In these treatments, a large amount of dialysate, for example about 120 liters, is consumed to dialyze the blood during a single hemodialysis therapy. Treatment can last several hours and may be performed in a treatment center about three or four times per week. In peritoneal dialysis (PD), patients must perform three to four dialysate fluid exchanges per day with continuous ambulatory PD or connect themselves to an automated overnight requiring fresh dialysate for every exchange. A PD system that purifies and recycles dialysate would reduce need to use high amount of dialysate solution, and fewer connections and disconnections could potentially reduce the risk of peritonitis.

It may be beneficial to lessen dialysate volume and/or improve treatment in dialysis systems.

### SUMMARY

According to one example (“Example 1”), a filter for removal of waste products from a dialysate includes multiple layers of one or more filter materials configured to allow dialysate to flow through the multiple layers; each filter material comprising a porous polyolefin that is filled with sorbent particles; and at least one layer of filter material including particles containing a urease.

According to another example (“Example 2”), further to the filter of Example 1, the one or more filter materials including additional particles to absorb creatinine.

According to another example (“Example 3”), further to the filter of any one of Examples 1-2, the one or more filter materials including additional particles to absorb heavy metals.

According to another example (“Example 4”), further to the filter of any one of Examples 1-3, the one or more filter materials including additional particles to absorb ammonia.

According to another example (“Example 5”), further to the filter of any one of Examples 1-4, the multiple layers include at least a first layer, a second layer, and a third layer with the second layer being arranged between the first layer and the third layer.

According to another example (“Example 6”), further to the filter of Example 5, the first layer is configured to hold molecules having a first diameter in response to the dialysate flowing through the multiple layers, the second layer is configured to hold molecules having a second diameter in response to the dialysate flowing through the multiple layers, and the third layer is configured to hold molecules having a third diameter in response to the dialysate flowing through the multiple layers.

According to another example (“Example 7”), further to the filter of Example 6, the first layer is configured to hold molecules by at least one of a physical reaction and a chemical reaction, the second layer is configured to hold molecules by at least one of a physical reaction and a chemical reaction, and the third layer is configured to hold molecules by at least one of a physical reaction and a chemical reaction.

According to another example (“Example 8”), further to the filter of Example 6, the first layer is configured to hold molecules by at least one of adsorption and absorption, the second layer is configured to hold molecules by at least one of adsorption and absorption, and the third layer is configured to hold molecules by at least one of adsorption and absorption.

According to another example (“Example 9”), further to the filter of Example 5, at least one of the first layer, the second layer, and the third layer is configured to convert the waste products to non-waste products in response to the dialysate flowing through the multiple layers.

According to another example (“Example 10”), further to the filter of Example 5, at least one of the first layer, the second layer, and the third layer is configured to hold molecules having a first diameter in response to the dialysate flowing through the multiple layers and hold molecules having a second diameter in response to the dialysate flowing through the multiple layers.

According to another example (“Example 11”), further to the filter of any one of Examples 1-10, the one or more filter materials including additional particles to absorb uric acid.

According to another example (“Example 12”), further to the filter of any one of Examples 1-11, the one or more filter materials including additional particles to absorb macroglobulins.

According to another example (“Example 13”), further to the filter of any one of Examples 1-12, the one or more filter materials including additional particles to absorb phosphates.

According to another example (“Example 14”), further to the filter of any one of Examples 1-13, the one or more filter materials including additional particles to absorb middle size organic waste molecules.

According to another example (“Example 15”), further to the filter of any one of Examples 1-14, the one or more filter materials including additional particles to absorb fluoride.

According to another example (“Example 16”), further to the filter of any one of Examples 1-15, wherein the one or more filter materials including additional particles to absorb chloramines.

According to another example (“Example 17”), further to the filter of any one of Examples 1-16, the polyolefin comprises PTFE.

According to another example (“Example 18”), further to the filter of any one of Examples 1-17, the polyolefin comprises PE.

According to another example (“Example 19”), further to the filter of any one of Examples 1-18, the multiple layers of one or more filter materials are mounted in a dialysis system.

According to one example (“Example 20”), a filter for removal of waste products from a dialysate includes multiple layers of one or more filter materials configured to allow dialysate to flow through them; each filter material comprising a porous polyolefin that is filled with sorbent particles; and at least one layer of filter material including particles containing a material for treatment of urea.

According to another example (“Example 21”), further to the filter of Example 20, the material for treatment of urea comprises urease.

According to another example (“Example 22”), further to the filter of Example 20, the material for treatment of urea effectuates physisorption of urea.

According to one example (“Example 23”), a filter for removal of waste products from a dialysate includes a filter cartridge configured to allow dialysate to flow through it, the cartridge containing multiple layers of one or more filter materials; each filter material comprising a porous polyolefin that is filled with sorbent particles; and at least one layer of filter material including particles containing a material for treatment of urea.

According to another example (“Example 24”), further to the filter of Example 23, the filter is configured such that the dialysate is regenerated or purified in a single pass through the filter cartridge.

According to one example (“Example 25”), a dialysis system that removes waste products through use of a dialysate includes a filter having multiple layers of one or more filter materials configured to allow dialysate to interact with the filter materials; each filter material comprising a porous polyolefin that is filled with sorbent particles; and at least one layer of filter material including particles containing a material for treatment of urea.

According to another example (“Example 26”), further to the filter of Example 25, the material for treatment of urea comprises urease.

According to another example (“Example 27”), further to the filter of Example 25, the porous polyolefin comprises ePTFE.

According to another example (“Example 28”), further to the filter of Example 25, the filter is housed within a filter cartridge configured to allow dialysate to flow through it.

According to another example (“Example 29”), further to the filter of Example 28, filter is configured such that the dialysate is fully regenerated in a single pass through the filter cartridge.

According to another example (“Example 30”), further to the filter of Example 25, the dialysis system comprises a portable dialysis system.

According to another example (“Example 31”), further to the filter of Example 30, the dialysis system comprises a wearable dialysis system.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a diagram of an example hemodialysis system and filter, in accordance with an embodiment;

FIG. 1B is a diagram of an example peritoneal system and filter, in accordance with an embodiment;

FIG. 2 is an illustration of an example filter and filter cartridge, in accordance with an embodiment;

FIG. 3 is an illustration of an example filter layer, in accordance with an embodiment; and

FIG. 4 is a scanning electron microcopy (SEM) image of an example filter layer, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are generally directed toward apparatuses, systems and methods that include a filter for removal of waste products from a spent dialysate. The filter, in certain instances, may be used in dialysis systems and maintain a high flow rate of the dialysate through the filter. The filter also may include a high capacity to effectively filter a large number (e.g., high absorption capacity as compared to prior filters) and a large variety of toxins from the dialysate. The filter may be used with an in-center dialysis system, a portable at-home dialysis system, or a wearable dialysis system.

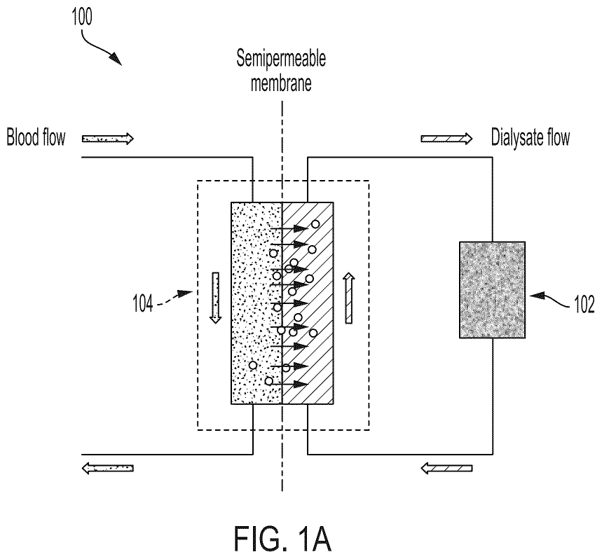

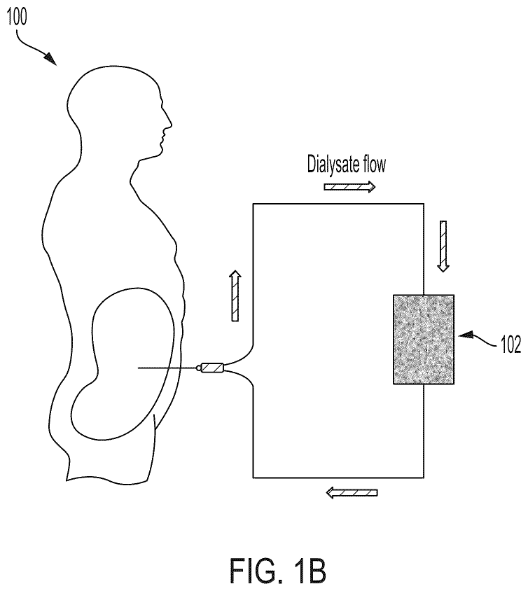

FIG. 1A is a diagram of an example hemodialysis system 100 and filter 102, in accordance with an embodiment. The system 100 may include one or more pumps (not shown) that drive blood and dialysate through the system 100. Blood and dialysate flow through the system 100 and are pumped through a dialyzer 104 where waste is removed from the blood and collected by the dialysate. The blood and dialysate flow in a continuous loop for continuous cleaning of a patient's blood. Rather than pumping new dialysate through the system, spent dialysate is passed through the filter 102 where the dialysate is cleansed and returned toward the dialyzer 104. The hemodialysis system may also contain one or more sensors, monitors, detectors, air traps, or fluid dispensers (not shown).

As discussed in further detail below, the filter 102 may include multiple layers of one or more filter materials configured to allow dialysate to interact with the filter materials. Each filter material comprising a porous polymer such as a porous polyolefin (e.g., ePTFE) that is filled with sorbent particles and at least one layer of filter material including particles. The particles may contain a material for treatment of urea (e.g., urease). In other instances, the filter 102 may include a packed bed of porous polymer (including filter material particles) or a functionalized porous polyolefin (e.g., PTFE) in addition or in alternative to particle filed porous polyolefin. The filter 102 may be housed within a filter cartridge configured to allow dialysate to flow through it. The filter 102 may be configured such that the dialysate is regenerated or purified in a single pass through the filter 102.

The filter 102, as discussed in further detail below, may include multiple layers of a porous polyolefin filled with particles that adsorb, absorb, or otherwise remove toxins from dialysate. The filter 102 may be highly efficient such that the dialysate may be recirculated through the system 100 after cleaning. In certain instances, the filter 102 may enable the system to use and re-use a smaller volume of dialysate, as compared to a traditional dialysis treatment, throughout a single or multiple dialysis treatment cycles. The filter 102, for example, may enable a reduction in volume of dialysate in the order of up to 25 times as compared to a traditional dialysis treatment. The filter 102 enabling a reduction in volume of the dialysate may greatly reduce cost, size, and weight of the system 100 as compared to a traditional dialysis treatment.

The filter 102 including multiple layers of a porous polyolefin filled with particles may enable a high capacity and highly efficient removal of toxins. The layers of the filter 102, for example, may be configured to adsorb, absorb, or otherwise remove different toxins. For example, one layer may be configured to adsorb, absorb, or otherwise remove large molecules while another layer may be configured to adsorb, absorb, or otherwise remove a different size or type of molecule from the dialysate. In addition, the filter 102 may include multiple filters 102 or cartridges that include the filter 102 that target different or similar toxins in the dialysate.

The filter 102 including multiple layers of a porous polyolefin facilitates a low pressure drop of flow of the dialysate through the system 100. The porous nature of the polyolefin maintains the flow of dialysate without slowing waste removal from the blood.

FIG. 1B is a diagram of an example peritoneal system 100 that incorporates the filter 102, in accordance with an embodiment. The peritoneal system 100 does not use a dialyzer and infuses dialysate into a patient. The filter 102 enables reduction in dialysate volume similar to the reduction discussed above. As opposed to new dialysate infused into the patient, the dialysate may be passed through the filter 102 and recirculated, which enables the reduction in dialysate volume. The peritoneal dialysis system may also contain one or more sensors, monitors, detectors, air traps, or fluid dispensers (not shown).

In either instance, the dialysis system 100 may be a portable dialysis system. The high capacity removal of toxins by the filter 102 may enable a large reduction in dialysate volume as noted above. The reduction in dialysate volume and size of the filter 102 enables the system 100 to be an in-home system or a wearable dialysis system in certain instances.

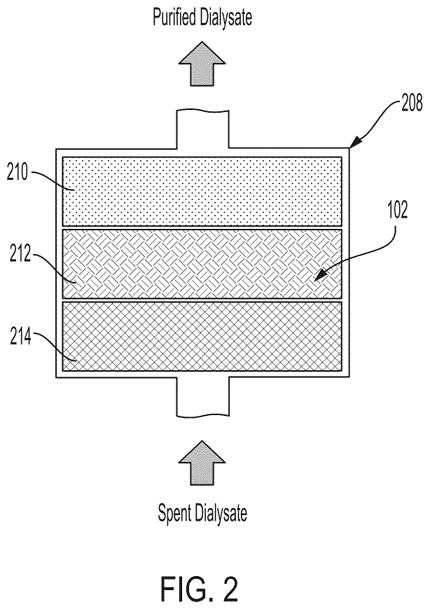

FIG. 2 is an illustration of an example filter 102 and filter cartridge 208, in accordance with an embodiment. As noted above, the filter 102 may be used for removal of waste products from a dialysate. As shown, the filter 102 includes multiple layers 210, 212, 214 and is arranged within the filter cartridge 208. The multiple layers 210, 212, 214 include one or more filter materials configured to allow dialysate to flow through the multiple layers 210, 212, 214 with each filter material having a porous polyolefin that is filled with sorbent particles. Further, at least one layer of filter 102 material includes particles that may contain a urease to clear urea enzymatically. Further, the one or more filter materials of the layers 210, 212, 214 may include additional particles to remove ammonia. Spent dialysate may flow into and through the filter cartridge 208, including each of the multiple layers 210, 212, 214 in the filter cartridge 208, with clean dialysate flowing out of the filter cartridge 208.

In certain instances, the one or more filter materials of the layers 210, 212, 214 include additional particles to absorb creatinine. In addition, the one or more filter materials of the layers 210, 212, 214 may include additional particles to absorb heavy metals.

The filter 102 may include one, two, three, four, five, six, seven, eight and above number of layers of a filter material having a porous polyolefin that is filled with sorbent particles. The layers may include the same or different sorbent particles. In certain instances, the multiple layers 210, 212, 214 of the filter 102 includes a first layer 210, a second layer 212, and a third layer 214. The second layer 212 is arranged between the first layer 210 and the third layer 214. As noted above, layers 210, 212, 214 may include the same or different sorbent particles. In certain instances, the first layer 210 is configured to hold molecules having a first diameter in response to the dialysate flowing through the multiple layers 210, 212, 214. The second layer 212 may be configured to hold molecules having a second diameter in response to the dialysate flowing through the multiple layers 210, 212, 214. In addition, the third layer 214 may be configured to hold molecules having a third diameter in response to the dialysate flowing through the multiple layers 210, 212, 214.

In certain instances, the first layer 210, the second layer 212, and/or the third layer 214 is configured to hold molecules having a first diameter in response to the dialysate flowing through the multiple layers and hold molecules having a second diameter in response to the dialysate flowing through the multiple layers. The first layer 210, the second layer 212, and/or the third layer 214 may also be configured to hold molecules having a third diameter in addition to molecules of the first and second diameter. One or more of the multiple layers 210, 212, 214 may have include particles arranged within a single layer that are configured to hold molecules of differing diameters.

In certain instances, the first layer 210 is configured to hold molecules by a physical reaction (e.g., adsorption), a chemical reaction (e.g., absorption), and/or the first layer 210 is configured to convert the waste products to non-waste products in response to the dialysate flowing through the first layer 210. In certain instances, the first layer 210 may include different particles that each separately hold molecules by a physical reaction (e.g., adsorption), a chemical reaction (e.g., absorption), and convert the waste products to non-waste products. In other instances, the first layer 210 may include a single type of particle such that the first layer 210 is configured to hold molecules by a physical reaction (e.g., adsorption), a chemical reaction (e.g., absorption), or the first layer 210 is configured to convert the waste products to non-waste products.

In certain instances, the second layer 212 is configured to hold molecules by a physical reaction (e.g., adsorption), a chemical reaction (e.g., absorption), and/or the second layer 212 is configured to convert the waste products to non-waste products in response to the dialysate flowing through the second layer 212. In certain instances, the second layer 212 may include different particles that each separately hold molecules by a physical reaction (e.g., adsorption), a chemical reaction (e.g., absorption), and convert the waste products to non-waste products. In other instances, the second layer 212 may include a single type of particle such that the second layer 212 is configured to hold molecules by a physical reaction (e.g., adsorption), a chemical reaction (e.g., absorption), or the second layer 212 is configured to convert the waste products to non-waste products.

In certain instances, the third layer 214 is configured to hold molecules by a physical reaction (e.g., adsorption), a chemical reaction (e.g., absorption), and/or the third layer 214 is configured to convert the waste products to non-waste products in response to the dialysate flowing through the third layer 214. In certain instances, the third layer 214 may include different particles that each separately hold molecules by a physical reaction (e.g., adsorption), a chemical reaction (e.g., absorption), and convert the waste products to non-waste products. In other instances, the third layer 214 may include a single type of particle such that the third layer 214 is configured to hold molecules by a physical reaction (e.g., adsorption), a chemical reaction (e.g., absorption), or the third layer 214 is configured to convert the waste products to non-waste products.

In certain instances, one or more filter materials including in one or more of the first layer 210, the second layer 212, and/or the third layer 214 may include additional particles to absorb uric acid. In addition, one or more filter materials including in one or more of the first layer 210, the second layer 212, and/or the third layer 214 may include additional particles to absorb macroglobulins. Further, the one or more filter materials including in one or more of the first layer 210, the second layer 212, and/or the third layer 214 may include additional particles to absorb phosphates. Further, one or more filter materials including in one or more of the first layer 210, the second layer 212, and/or the third layer 214 may include additional particles to absorb middle size organic waste molecules. One or more filter materials including in one or more of the first layer 210, the second layer 212, and/or the third layer 214 may include additional particles to absorb fluoride in certain instances. The one or more filter materials including in one or more of the first layer 210, the second layer 212, and/or the third layer 214 may include additional particles may also be configured to absorb chloramines. In addition, the one or more filter materials including in one or more of the first layer 210, the second layer 212, and/or the third layer 214 may include urease. In certain instances, the one or more filter materials including in one or more of the first layer 210, the second layer 212, and/or the third layer 214 may effectuate physisorption of urea.

In certain instances, the first layer 210, the second layer 212, and the third layer 214 may be arranged such that certain molecules are held or cleansed prior to other molecules. For example, the first layer 210 may be configured to hold a first type of molecule, the second layer 212 may be configured to hold a second type of molecule, and the third layer 214 may be configured to hold a third type of molecule. In certain instances, the first layer 210, the second layer 212, and the third layer 214 may be arranged such that larger molecules are filtered prior to filtering of smaller molecules. For example, the third layer 214 may be configured to hold larger molecules than the second layer 212, and the second layer 212 may be configured to hold larger molecules than the first layer 210. In other instances, the first layer 210, the second layer 212, and the third layer 214 may be arranged such that smaller molecules are filtered first. For example, the third layer 214 may be configured to hold smaller molecules than the second layer 212, and the second layer 212 may be configured to hold smaller molecules than the first layer 210.

The multiple layers 210, 212, 214 may be formed within the filter cartridge 208 such that the multiple layers 210, 212, 214 are arranged in a stacked layered configuration or arranged in a spiral tape configuration. In this manner, the multiple layers 210, 212, 214 may conform to the shape of the filter cartridge 208. As noted above, the filter cartridge 208, and the multiple layers 210, 212, 214 of one or more filter materials, are mounted in a dialysis system (such as the system 100 shown in FIGS. 1A-B). The filter 102 may be configured such that the dialysate is purified or regenerated in a single pass through the filter cartridge 208.

The system may recirculate the dialysate through the filter 102 enabling a reduction in volume of dialysate used (5-10 liters as compared to upwards of 120 liters). In dialysis center, a treatment session may be 3-4 hours and 3-4 sessions a week. A system using the filter 102 may enable steady treatment over a length of time (e.g., in a patient's sleep). The steady treatment may facilitate consistent filtering of toxins and lessen time between treatments that occur when a patient is in a dialysis center.

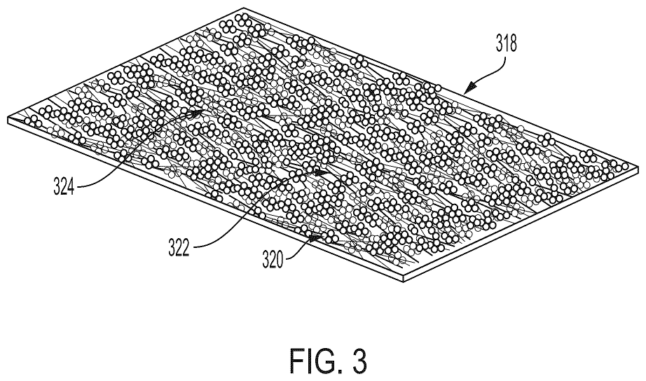

FIG. 3 is an illustration of an example filter layer 318, in accordance with an embodiment. The filter layer 318 may be used as one or more layers that is incorporated into a filter for removal of waste products from a dialysate as discussed in detail above. The filter layer 318 may include a porous polyolefin that is filled with sorbent particles 320. In certain instances, the polyolefin may be PTFE. The polyolefin may also be PE.

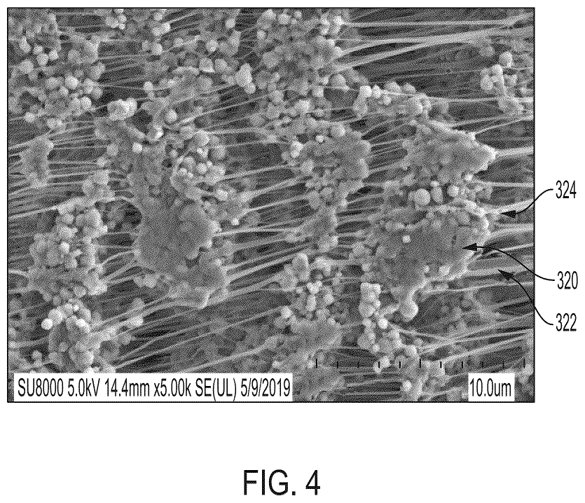

The filter layer 318 being formed of polyolefin may be advantageous in that polyolefin includes micropores formed between polymer fibrils 322 and nodes 324. The sorbent particles 320 may be contained within the pores between the polymer fibrils 322 and nodes 324. FIG. 4 is a scanning electron microcopy (SEM) image of an example filter layer showing the sorbent particles 320 contained within the pores between the polymer fibrils 322 and nodes 324.

Suitable materials that is suitable for use in filter materials include, but are is not limited to, polyolefin, microporous polyethylene, and expanded fluoropolymer membranes such as expanded polytetrafluoroethylene (ePTFE) or other porous synthetic polymer materials. Such filter materials can comprise PTFE homopolymer, blends of PTFE, expandable modified PTFE and/or expanded copolymers of PTFE. As referenced, the filter materials may have a microporous structures (e.g., such as ePTFE materials including a matrix of fibrils defining a plurality of spaces within the matrix).

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A filter for removal of waste products from a dialysate, the filter comprising:
multiple layers of one or more filter materials configured to allow dialysate to flow through the multiple layers;
each filter material comprising a porous polyolefin that is filled with sorbent particles; and
at least one layer of filter material including particles containing a urease.

2. The filter of claim 1, wherein the one or more filter materials including additional particles to absorb creatinine.

3. The filter of claim 1, wherein the one or more filter materials including additional particles to absorb heavy metals.

4. The filter of claim 1, wherein the one or more filter materials including additional particles to absorb ammonia.

5. The filter of claim 1, wherein the multiple layers include at least a first layer, a second layer, and a third layer with the second layer being arranged between the first layer and the third layer.

6. The filter of claim 5, wherein the first layer is configured to hold molecules having a first diameter in response to the dialysate flowing through the multiple layers, the second layer is configured to hold molecules having a second diameter in response to the dialysate flowing through the multiple layers, and the third layer is configured to hold molecules having a third diameter in response to the dialysate flowing through the multiple layers.

7. The filter of claim 6, wherein the first layer is configured to hold molecules by at least one of a physical reaction and a chemical reaction, the second layer is configured to hold molecules by at least one of a physical reaction and a chemical reaction, and the third layer is configured to hold molecules by at least one of a physical reaction and a chemical reaction.

8. The filter of claim 6, wherein the first layer is configured to hold molecules by at least one of adsorption and absorption, the second layer is configured to hold molecules by at least one of adsorption and absorption, and the third layer is configured to hold molecules by at least one of adsorption and absorption.

9. The filter of claim 5, wherein at least one of the first layer, the second layer, and the third layer is configured to convert the waste products to non-waste products in response to the dialysate flowing through the multiple layers.

10. The filter of claim 5, wherein at least one of the first layer, the second layer, and the third layer is configured to hold molecules having a first diameter in response to the dialysate flowing through the multiple layers and hold molecules having a second diameter in response to the dialysate flowing through the multiple layers.

11. The filter of claim 1, wherein the one or more filter materials including additional particles to absorb uric acid.

12. The filter of claim 1, wherein the one or more filter materials including additional particles to absorb macroglobulins.

13. The filter of claim 1, wherein the one or more filter materials including additional particles to absorb phosphates.

14. The filter of claim 1, wherein the one or more filter materials including additional particles to absorb middle size organic waste molecules.

15. The filter of claim 1, wherein the one or more filter materials including additional particles to absorb fluoride.

16. The filter of claim 1, wherein the one or more filter materials including additional particles to absorb chloramines.

17. The filter of claim 1, wherein the polyolefin comprises PTFE.

18. The filter of claim 1, wherein the polyolefin comprises PE.

19. The filter of claim 1, wherein the multiple layers of one or more filter materials are mounted in a dialysis system.

20. A filter for removal of waste products from a dialysate, the filter comprises:
multiple layers of one or more filter materials configured to allow dialysate to flow through them;
each filter material comprising a porous polyolefin that is filled with sorbent particles; and
at least one layer of filter material including particles containing a material for treatment of urea.

21. The filter of claim 20, wherein the material for treatment of urea comprises urease.

22. The filter of claim 20, wherein the material for treatment of urea effectuates physisorption of urea.

23. A filter for removal of waste products from a dialysate, the filter comprising:
a filter cartridge configured to allow dialysate to flow through it, the cartridge containing multiple
layers of one or more filter materials;
each filter material comprising a porous polyolefin that is filled with sorbent particles; and
at least one layer of filter material including particles containing a material for treatment of urea.

24. The filter of claim 23, wherein the filter is configured such that the dialysate is regenerated or purified in a single pass through the filter cartridge.

25. A dialysis system that removes waste products through use of a dialysate, the system comprising:
a filter having multiple layers of one or more filter materials configured to allow dialysate to interact with the filter materials;
each filter material comprising a porous polyolefin that is filled with sorbent particles; and
at least one layer of filter material including particles containing a material for treatment of urea.

26. The dialysis system of claim 25, wherein the material for treatment of urea comprises urease.

27. The dialysis system of claim 25, wherein the porous polyolefin comprises ePTFE.

28. The dialysis system of claim 25, wherein the filter is housed within a filter cartridge configured to allow dialysate to flow through it.

29. The dialysis system of claim 28, wherein filter is configured such that the dialysate is fully regenerated in a single pass through the filter cartridge.

30. The dialysis system of claim 25, wherein the dialysis system comprises a portable dialysis system.

31. The dialysis system of claim 30, wherein the dialysis system comprises a wearable dialysis system.
